(19) United States
(12) Patent Application Publication
(10) Pub. No.: US 2014/0106002 A1
(43) Pub. Date: Apr. 17, 2014

(54) HOMEOPATHIC COMPOSITION AND METHOD FOR THE TREATMENT OF SKIN IRRITATIONS AND OTHER SKIN DISEASES

(71) Applicants: Ned L. Jensen, Alpine, UT (US); Peter E. Fuller, West Jordan, UT (US)

(72) Inventors: Ned L. Jensen, Alpine, UT (US); Peter E. Fuller, West Jordan, UT (US)

(21) Appl. No.: 13/841,902

(22) Filed: Mar. 15, 2013

Related U.S. Application Data
(63) Continuation-in-part of application No. 13/113,226, filed on May 23, 2011, now abandoned.

(51) Int. Cl. A61K 36/896; A61K 33/00; A61K 31/045; A61K 36/185; A61K 36/53; A61K 36/28; A61K 33/30; A61K 33/38; A61K 36/537; A61K 31/60; A61K 36/29

(52) U.S. Cl. CPC A61K 36/896; A61K 31/60; A61K 33/00; A61K 31/045; A61K 36/185; A61K 36/29; A61K 36/28; A61K 33/30; A61K 33/38; A61K 36/537; A61K 36/53
USPC 424/618; 424/722; 424/641; 424/58; 424/49

(57) ABSTRACT

A homeopathic composition and method for treating a variety of skin irritations, wounds, and diseases is disclosed. The homeopathic composition may contain as active ingredients salicyclic acid, trona or one of its salts, and at least one homeopathic agent.

This application is a continuation-in-part of pending U.S. patent application Ser. No. 13/113,226 filed May 23, 2011.

### BACKGROUND OF THE INVENTION

The present invention generally relates to compositions and methods for the treatment of skin irritations and internal ailments. More specifically, the present invention relates to compositions and methods for treating skin and internal ailments by topically or orally administering a therapeutically effective amount of a homeopathic composition containing salicyclic acid, akaline earth carbonates and bicarbonates, and one or more homeopathic agents.

In recent years, there has been an increased effort to reduce or eliminate the risk of sunburn (erythema) produced by certain wavelengths in the ultraviolet (UV) region of the spectrum. Unfortunately, there are still circumstances wherein the skin becomes exposed to UV radiation. Such exposure may, in some cases, cause sunburn that needs to be treated.

The application of petroleum-based compositions to the sunburned area does not produce a sufficiently large heat transference effect to remove the heat from the sunburned area. Furthermore, the petroleum-based compositions tend to produce a residue that needs to be subsequently cleansed from the tender and sensitive area of sunburned skin, causing further discomfort to the patient.

As can be seen, there is a need for a composition for treating sunburn that provides immediate pain relief to the patient while also helping to promote healing of the skin.

### SUMMARY OF THE INVENTION

In one aspect the present invention is drawn to a homeopathic composition for the treatment of skin irritations and other ailments comprising salicylic acid, trona or a trona salt, and at least one homeopathic agent.

In another aspect the present invention is drawn to a homeopathic composition for the treatment of skin irritations and other ailments comprising salicylic acid, trona, and one or more homeopathic agents.

Preferred homeopathic agents or ingredients are those selected from the group consisting of: Aesculus Hippocastum (Horse Chestnut), Berberis Vulgaris (Barberry), Calendula Officianalis (Marigold), Aloe Vera, Salvia Officinalis (Sage), Thyme, Menthol, Zinc Metallicum or Salts 2× to 6×, Arnica Montana, Silver (Argentum Metallicum) or Salts 4× to 6×, Thyme, Potassium Chloride, Sodium Chloride, Phosphorus 3× to 12×, Sulphur OX to 3×, Manganese Metallicum or Salts 2× to 6×, Copper Metalicum or Salts 3× to 6×, Clove, Golden Seal (Hydrastis), Witch Hazel, Wintergreen, Frankencense, Myrrh, Eucalyptus, White Willow (natural source of salicylic acid), Rosemary, Lavender, Lemon, Lime, Orange, Arsenicum Album, Arsenicum Trioxide 5× to 12×, and Mercury Metalicum or Salts 5× to 12×.

Particularly preferred homeopathic agent or ingredients are those selected from the group consisting of Aesculus Hippocastum (Horse Chestnut), Berberis Vulgaris (Barberry), Calendula Officianalis (Marigold), Aloe Vera, Salvia Officinalis (Sage), and Thyme.

In still another aspect, the present invention is drawn to a method for treating a skin irritation or other ailments in a patient including the step of topically administering to the affected area a therapeutically effective amount of a homeopathic composition comprising a salicylic acid; trona or a trona salt, and at least one homeopathic agent.

In another aspect, the present invention is drawn to a method for treating a skin irritation or other ailments in a patient including the step of topically administering to the affected area a therapeutically effective amount of a homeopathic composition comprising a salicylic acid; trona and one or more homeopathic agents selected from the group consisting of one or more of the above listed preferred or most preferred homeopathic agents or ingredients.

In a yet different aspect, the present invention is drawn to a method for treating ailments in a patient including the step of oral administering to the patient a therapeutically effective amount of a homeopathic composition comprising a salicylic acid; trona or a trona salt, and at least one homeopathic agent.

In a subsequent aspect, the present invention is drawn to a method for treating ailments in a patient including the step of orally administering to the patient a therapeutically effective amount of a homeopathic composition comprising a salicylic acid; trona and one or more homeopathic agents selected from the group consisting of one or more of the above listed preferred or most preferred homeopathic agents or ingredients.

In a still different aspect, the present invention is drawn to a method for maintaining the freshness of a product including the steps of adding to an environment surrounding the product a homeopathic composition comprising salicylic acid; trona or one of its salts; and at least one homeopathic agent.

Again, another aspect of the present invention is drawn to a method for maintaining the freshness of a product including the steps of adding to an environment surrounding the product a homeopathic composition comprising salicylic acid, trona and one or more homeopathic agents selected from the group consisting of one or more of the above listed preferred or most preferred homeopathic agents or ingredients.

These and other features, aspects and advantages of the present invention will become better understood with reference to the following drawings, description and claims.

### BRIEF DESCRIPTION OF THE DRAWINGS

FIG. 1 illustrates a schematic diagram showing active ingredients used to prepare the composition according to an exemplary embodiment of the present invention; and

FIGS. 2-3 illustrate different bases or devices which are also representative of the compositions which may be administered according to exemplary embodiments of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features used independently of one another or in combination with other features.

Broadly, embodiments of the present invention generally provides homeopathic compositions for treating a variety of skin irritations, wounds, internal ailments and diseases by administering topically to the affected area, or orally if to be used for an internal ailment, a therapeutically effective amount of a homeopathic composition containing as active ingredients salicyclic acid, one or more homeopathic agents, and trona or one or more trona salts.

As used herein, the term “skin irritation” may refer to any skin inflammation or itchiness causing discomfort to the patient. The skin irritation may be caused by burns, such as sunburn, heat burn, poisonous plants, allergens, insect bites, or puberty (acne); and wounds, such as lacerations.

As used herein the term “internal ailment” may refer to any internal affliction including, but not limited to, headache, stomach ache, acid reflux, backache, skin rashes, arthritis, neuralgia, and joint pain, for which oral administration is indicated.

As used herein, the term “therapeutically effective amount” is intended to refer to the amount of the composition necessary to bring relief to any affected external or internal tissue(s), organ(s), or organ system(s) of the patient when topically or orally administered.

As used herein, the term “topically administering” is intended to refer to the direct laying on, or spreading of, a composition on epidermal tissue at the affected site.

As used herein, the term “orally administering” is intended to refer to the direct administration by mouth to the affected individual.

As used herein, the term “homeopathic composition” is intended to refer to a composition for treating a disease based on the administration of minute doses of the agent that in massive amounts produces symptoms in healthy individuals similar to those of the disease itself. Thus, after a base preparation is made, either by an extract or maceration of an herbal compound or the dissolving of a selected compound in a solvent, a series of dilutions are prepared from the initial batch, sometimes called the “mother tincture.” Tincture nomenclature is logarithmic, when a concentration indicates 3×, the substance has been diluted at a factor of 1,000 (10³). Another example include 6× or a dilution factor of 1,000,000 (10⁶), etc. Preparation of the finished product further dilutes the homeopathic substance.

Homeopathic compositions may be diluted according to either the decimal “xn” or centesimal “C” scales. The “xn” scale is used herein. For a “3×HPUS” preparation, the mother tincture (1×) is diluted with nine parts of the desired diluent, in either liquid or powder form (2×). The resultant mixture is then diluted a second time, in a ratio of one part mixture to nine parts solvent and the resulting mixture results in a 3× dilution. Therefore, the 3× drug is actually at 10³ potency of the herbal ingredient. These scales are recognized by the Homeopathic Pharmacopeia of the United States (H.P.U.S.).

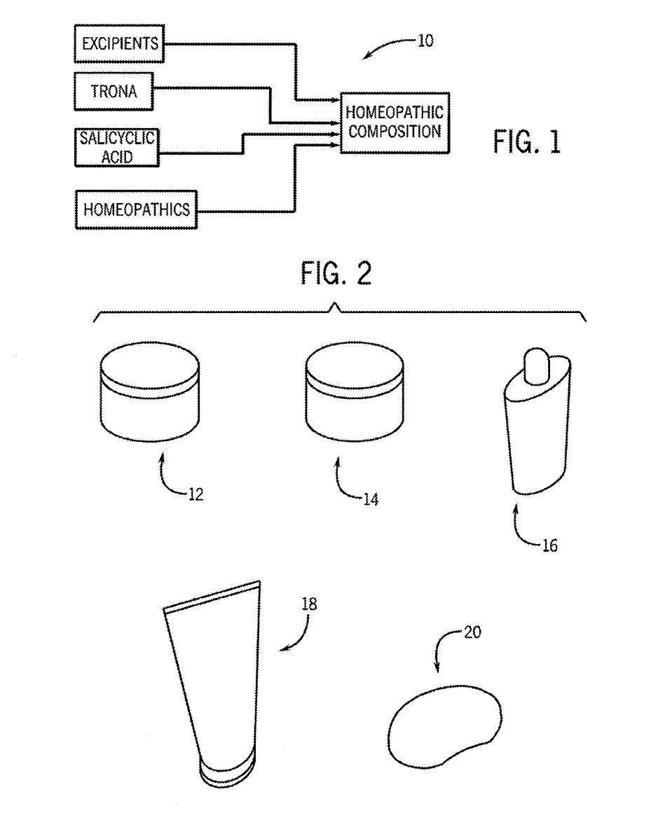

FIG. 1 illustrates, or is representative of, active ingredients for a homeopathic composition according to an exemplary embodiment of the present invention. The homeopathic composition 10 may include as active ingredients trisodium hydrogendicarbonate dihydrate (Trona), salicyclic acid, and at least one homeopathic agent as well as excipients, carriers, and the like more commonly referred to herein as a “base”.

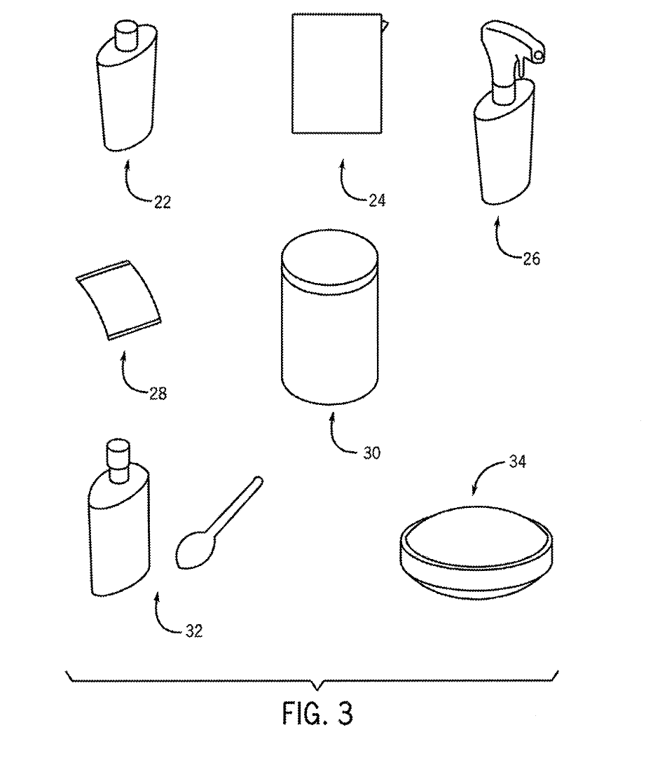

The homeopathic composition 10 may be incorporated into a mixture or composition for administration purposes. FIGS. 2-3 illustrate that the composition housed or contained in the device may be a cream 12, a paste 14, a liquid 16, a gel 18, a solid 20, a lotion 22, a powder 24, a spray 26, an ointment 28, an emulsion 30, oral solution 32, tablets 34, or capsules (not shown). The cream 12 may include, hand cream, shaving cream, antibacterial cream, acne cleanser, facial cleaning cream, or body cream. The paste 14 may include toothpaste, acne scrub, or facial cleansers. The liquid 16 may include liquid soaps, liquid detergents, liquid bubble bath soaps. food preservatives, face cleansers, food additives, liquid laundry detergent, or food preservation agents. The gel 18 may include toothpaste, acne topical gel, air refresher in gel form, food preservation agents, face cleansers, or soaps in gel form. The solid 20 may include soap, capsules, or pills. The lotion 22 may include body lotions, face cleansers, acne lotions, or shaving lotions. The powder 24 may include detergents. plant food additives, or food preservatives.

In its broadest sense, trona is a natural mineral composed mainly of sodium carbonate, sodium bicarbonate, potassium carbonate and potassium bicarbonate with small amounts of magnesium and calcium salts and other trace minerals. Trona, when used herein, refers to trisodium hydrogendicarbonate dehydrate, having the formula: Na₃(CO₃)(HCO₃).2H₂O. When the term “and salts thereof” or “composite salts” follows “trona” it is meant to include the broader definition including the natural mineral salts thereof inclusive of potassium, magnesium and calcium salts. The homeopathic compositions of this invention may use any commercially available form of trona including trona composite salts with trona or trisodium hydrogendicarbonate dehydrate, having the formula: Na₃(CO₃)(HCO₃).2H₂O, being preferred.

As noted above, the homeopathic composition 10 may use any commercially available form of trona including trona composite salts. As also noted, trona, as described herein, is preferred. The trona may be added to the homeopathic composition 10 for promoting the healing of the skin. The amount of trona in the homeopathic composition 10 may depend on the type of skin irritation to be treated. In some embodiments, the amount of trona present in the homeopathic composition 10 may be between 0.1 and 10% weight of the total composition 10. In some embodiments, the amount of trona present in homeopathic composition 10 may be 2.5% weight of the total homeopathic composition 10.

Salicylic acid may provide the homeopathic composition 10 with exfoliation and germicidal properties. Salicylic acid is an organic acid containing both —OH and —COOH groups may chelate minerals such as iron, manganese and copper. This chelating ability may give the salicylic acid the ability to act as a cleaning agent when combined with trona, various homeopathies, and sudsing agents. The amount of salicyclic acid in the homeopathic composition 10 may depend on the type of skin irritation to be treated. In some embodiments, the amount of salicylic acid present in the homeopathic composition 10 may be between 0.1 and 10% weight of the total homeopathic composition 10.

Salicylic acid may irritate the skin of the patient, but when combined with trona may produce a salt that may have a neutral pH that is gentle on the skin.

Homeopathic ingredients may be added to the homeopathic composition 10 to provide antibacterial, antifungal and antiviral activities. Homeopathic ingredients may include at least one of the preferred or most preferred homeopathic agents listed above. In addition, the homeopathic composition 10 may include as a homeopathic ingredient any homeopathic ingredient as outlined in the Homeopathic Pharmacopoeia of the United States and the British, German and Indian Homeopathic Pharmacopoeias.

In general a preparation to administer to the skin or orally may contain 1% to 10% trona or a trona salt, 1% to 10% salicylic acid and 1× to 2×% of one or more homeopathic ingredients

Carriers or bases may be added to the composition 10. The carriers or bases may be water, glycerin, ethanol, propylene glycol, isopropyl alcohol, vegetable oils such as soybean, avocado, sunflower, sesame seed oil, peanut oil, or other nut oils, canola, almond, jojoba oil, olive oil, safflower oil, grape seed oil, cranberry seed oil, sea buckthorn oil, any fatty acid ester such as isopropyl myristate, capryllic alcohol and esters.

In addition to salicylic acid, trona or a trona sale, and one or more homeopathic agents or ingredients the composition of the invention may, optionally, further comprise at least one of acidifying agents, alkalizing agents, aerosol propellants, antimicrobial agents, antioxidants, buffering agents, chelating agents, coloring additives, dispersing agents, emollients, emulsifying agents, humectants, fragrances, masking agents, preservatives, sugars, sunscreen agents, surfactants, suspending agents, or thickening agents.

Acidifying and alkalizing agents may be added to obtain the desired pH of the homeopathic composition 10. The acidifying agents may be at least one of acetic acid, citric acid, glacial acetic acid, malic acid, or proprionic acid. The alkalizing agents may be edetates, potassium carbonate, potassium hydroxide, sodium borate, sodium carbonate, or sodium hydroxide.

Aerosol propellants may be used when the homeopathic composition 10 may be administered as an aerosol under pressure. The aerosol propellants may be an inert gas including, but not limited to, nitrogen, carbon dioxide, helium, or argon.

Anti-microbial agents may be used when the skin area to be treated may be prone to microbial infection, bacteria, fungal, or protozoa. Anti-microbial agents may be benzyl alcohol, potassium sorbate, sorbic acid, benzoic acid, butyl paraben, ethyl paraben, methyl paraben, propyl pareben, or sodium benzoate.

Antioxidants may be used to protect ingredients of the homeopathic composition 10 from oxidizing agents that are included within or come in contact with the composition 10. The antioxidants may be at least one of water-soluble antioxidants such as all poly phenols, propyl gallate, ascorbic acid, carnosine, N-acetyl-carnosine, and cysteine hydrochloride and oil-soluble antioxidants such as retinoids, retinoic acid, all carotenes, ascorbyl palmitate, bulytlated hydroxyanisole, butylated hydroxytoluene, octyl gallate, dodecyl gallate, phenylalpha-napthyl-amine, tocotrienols, and tocopherols such as alpha-tocopherol. Buffering agents may be used to maintain an established pH for the composition 10. The buffering agents may be at least one of sodium citrate, calcium acetate, potassium metaphosphate, potassium phosphate monobasic, or tartaric acid.

Chelating agents may be used to maintain the ionic strength of the homeopathic composition 10 and/or bind to destructive compounds and metals that may be included within or come in contact with the homeopathic composition 10. The chelating agents may be at least one of dihydroxy ethyl glycine, amino acids, citric acid, tartaric acid, edetate dipotassium, edetate disodium, edetic acid, or ethylenediamine tetracetic acid (EDTA) and its salts (e.g., tetrasodium EDTA).

Coloring additives may be used to add color to the homeopathic composition 10. Coloring additives may be titanium dioxide, yellow iron oxide, red iron oxide, black iron oxide, caramel, carmine, fluorescein derivatives, methoxsalen, trioxsalen, carbon black, azo dyes, anthraquinone dyes, blue azulenes, guajazulene, chamuzulene, erythrosin, bengal rose, phloxin, cyanosin, daphinin, eosin G, cosin 108, or Acid Red 51 or any of the FD&C approved colors designated for food and cosmetic use.

Dispersing and suspending agents may be quarternium-18 hectorite, polyhydroxy stearic acid, magnesium stearate, or other fatty acid salts, poligeenan, or silicon dioxide.

Emollient agents may soften and smooth the skin. The emollients may be at least one of hydrocarbon such as mineral oil, petrolatum, microcrystalline wax, polyethylene, triglyceride esters such as those of castor oil, cocoa butter, safflower oil, cottonseed oil, corn oil, olive oil, cod liver oil, almond oil, avocado oil, palm oil, sesame oil, squalene, and soybean oil, acetylated monoglycerides, ethoxylated glycerides, fatty acids, alkyl esters of fatty acids, alkenyl esters of fatty acids, fatty alcohols, fatty alcohol ethers, ether-esters, lanolin and derivatives of lanolin, polyhydric alcohol esters, wax esters such as beeswax, vegetable waxes, phospholipids, or sterols.

Emulsifying agents may be used when the homeopathic composition 10 is an emulsion. The emulsifying agents used for preparing water-in-oil emulsions may be cyclomethicone (and) dimethicone copolyol, dimethicone copolyol, cetyl dimethicone copolyol, PEG-30 dipolyhydroxystearate, or PEG-40 sorbitan peroleate. The emulsifying agents used for preparing oil-in-water emulsions may be glyceryl stearate, PEG-100 stearate, methyl gluceth sesquisterate, fatty alcohols, or alkyl phenols condensed with ethylene oxide.

Humectants may promote the retention of moisture, e.g., moisturizers. The humectants may be at least one of sorbitol, matricaria extract, aloe barbadensis gel, glycerin, glycereth 5 lactate, glycereth 7 triacetate, glycereth 7 diisononoate, hexanetriol, hexylene glycol, propylene glycol, dipropylene glycol, alkoxylated glucose, D-panthenol, 1-2-pantandiol, 2-methyl1,3-propanediol, and derivatives thereof, or hyaluronic acid.

Fragrances may be at least one of peppermint, rose oil, rose water, aloe vera, clove oil, menthol, camphor, or eucalyptus oil. Certain fragrances may require a solubilizer, e.g., PPG-5-ceteareth-20. To eliminate certain odors from compositions, masking agents may be used. An example of a masking agent may be ethylene brassylate.

Preservatives may be used to protect the homeopathic composition 10 from degradation. The preservatives may be phenoxyethanol, methyl paraben, propyl paraben, butyl paraben, isopropyl paraben, isobutyl paraben, dieizolidinyl urea, imidazolidinyl urea, diazolindyl urea, benzalkonium chloride, benzethonium chloride, or phenol.

Sugars used may be at least one of monosaccharides, disaccharides, and polysccharides such as glucose, xylose, fructose, reose, ribose, pentose, arabinose, allose, tallose, altrose, mannose, galactose, lactose, sucrose, erythrose, or glyceraldehyde.

Sunscreen agents may be added to the homeopathic composition 10 to block or reduce the amount of ultraviolet radiation impinging on the skin. The sunscreen agents may be organic compounds and/or inorganic materials. The organic compounds may be at least one of oetyl methoxycinnamate, oetyl salicylate, benzophenone-3 homosalate, octocrylate, avobenzone, or menthyl anthranilate. The inorganic materials may be at least one of zinc oxide, silica, iron oxide, titanium dioxide, or 2-ethyl-hexyl-p-methoxycinnamate.

Surfactants may be used to stabilize the homeopathic compositions 10. The surfactants may be used as wetting agents, antifoam agents, emulsifiers, dispersing agents, or penetrant agents. The surfactants may include at least one of methyl gluceth 20, decyl polyglucoside, lapyrium chloride, laureth 4, laureth 9, monoethanolamine, nonoxynol 4, nonoxynol 9, nonoxynol 10, nonoxynol 15, nonoxynol 30, poloxalene, polyoxyl 8, 40, and 50 stearate, polysorbate 20, polysorbate 40, polysorbate 60, polysorbate 65, polysorbate 80, polysorbate 85, sodium lauryl sulfate, or sorbitan and its derivatives.

At least one humectant may be included when the homeopathic composition 10 is an aqueous composition. The humectant may reduce the tackiness produced by the aqueous composition after it has been applied and has dried on the skin. The humectants may be present in an amount such as to produce an aqueous composition that is capable of being substantially non-tacky after drying on the skin. The humectants may be at least one of glycerin, cellulose, or poly saccarides. The amount of humectants on the homeopathic composition 10 may be from about 0.1 to about 20% weight of the composition 10. In some embodiments, the amount of humectants on the homeopathic composition 10 may be 5% weight.

The ingredients may be mixed and/or blended together by using standard techniques well known in the art in order to manufacture the composition 10.

After mixing all of the ingredients, the homeopathic composition 10 may be packaged into the appropriate container so that the composition may be conveniently delivered and applied when needed. As illustrated in FIGS. 2 and 3, the container may be a tube, a bottle, a single-dose packet, box, sheet, or sprayer. In addition, the diluent composition 10 may be packed in” single-dose packages to reduce the risk of contamination from one usage to the next.

The examples that follow are representative of the invention but are not considered to be limiting. The invention is limited only by the claims and equivalent ingredients named therein. In the examples that follow concentrations are given in percent weight (% w) or (%) unless otherwise stated.

### Example 1

### Use of Aqueous Composition to Treat Sunburn

A Sunburn Spray as shown in the following table was prepared by combining all ingredients in a stainless steel tank and stirring until all ingredients went into solution and the mixture was homogenous.

| Type of | | | Active | Mgs or | |
|---|---|---|---|---|---|
| Ingredient | Ingredient Name | Latin Name | Agent | IU's | |
| | Glycerine DSP | | | 300 | mg |
| | Propylene Glycol | | | 300 | mg |
| | Deionized Water | | | 240 | mg |
| Homeopathic | Menthol | Mentholum | Same | 30 | mg |
| Homeopathic | Salicylic Acid | Salicylicum | Same | 30 | mg |
| | | Acidum | | | |
| Homeopathic | Aesculus | Same | Aesculin | 1 | mg |
| | Hippocastum 1x | | | | |
| Homeopathic | Berberis | Same | Berberine | 1 | mg |
| | Vulgaris 1x | | | | |
| Homeopathic | Calendula | Same | Calendulin | 1 | mg |
| | Officianalis 1x | | | | |
| Homeopathic | Aloe Vera 1x | Same | Barbaloin | 1 | mg |
| | Trona 10% | | Bicarbonate | 60 | ml |
| | Solution | | | | |
| Homeopathic | Zinc Sulfate | Zincum | Zinc | 5 | mg |
| | | Sulfuricum | | | |
| | Disodium EDTA | | Preservative | 6 | mg |
| | Benzyl Alcohol | | Preservative | 6 | mg |

This Sunburn Spray may be applied to the skin surface before going into the sun or after the skin has been burned. Results show that this spray cools the skin and kills the pain within a very short period of time, in some cases in about 15 seconds.

More specifically a 19-year old male and a 17-year old female, each suffering from classical erythema of the arms and face, were subjected to topical administration of the Sunburn Spray and their skin surface temperatures were monitored for changes. The temperatures were measured by using a Cooper Laser Thermometer. The initial skin surface temperatures prior to administration of the spray were:

(a) on the arms for the male—96° F. and for the female—94° F.

(b) on the face for the male—98° F. and for the female—96° F.

The skin surface temperatures about 90 seconds following administration of the homeopathic sunburn spray were:

(c) on the arms for the male-86° F. and for the female-86° F.

(d) on the face for the male-85° F. and for the female-85° F.

The results show a drop of over 10° F. in a minute and a half when using the Sun Spray. In addition, it was noted that the pain on the area of the skin where the Sun Spray was applied was completely reduced in the same period and the skin was not sensitive to touch.

It is believed that the decrease in skin surface temperature may be associated with the production of carbon dioxide by the reaction of salicyclic acid with the akaline earth carbonates and bicarbonates of trona in the presence of water. Carbon dioxide is a greenhouse gas, and is therefore effective for absorbing heat. Salicylic acid has anti-inflammatory properties which lead to a chemical change in the skin which may reduce the amounts of prostaglandins produced by cyclo-oxigenase in the skin.

It is also believed that the net result of combining salicyclic acid with the akaline earth carbonates and bicarbonates of the trona produces a composition with cooling properties.

### Example 2

### Cream Based Formula

A cream based formula may be made which also reduces inflammation and promotes healing and circulation. The cream based formula may contain appropriate amounts of deionized water, cetyl alcohol, glyceryl monostearate, isopropyl myristate, stearyl alcohol. avocado oil. sunflower oil. propylene glycol, glycerine, Vitamin E. lipoic acid, sodium benzoate, potassium sorbate, urea USP, polysorbate 20. xanthan gum. appropriate fragrances and MSM. To this crème base is added the active ingredients of salicylic acid 5%, Trona 2.5%, and homeopathies Aloe Vera 3×, Arnica 4×, Calendula 4×, Aesculus Hipp 4×, Berberis VulgariS 4×, Zinc Sulfuricum 3×.

The patients who applied this cream based formulation, to treat areas of the skin with burns and cuts, noted an immediate reduction of the pain, stopping of the bleeding, and reduction of the redness.

Some patients also note an increasing sensation of feeling and circulation when the cream based formulation of the present invention is applied to their feet.

### Example 3

### Antibacterial Cream

To the cream of Example 2 is added silver and zinc at 4×,i.e. at 100,000 or 10⁴ PPM to yield a cream with marked antibacterial activity.

### Example 4

### Acne Scrub

The sunburn spray formulation was modified to further include 10% cosmetic grade pumice, silver at 4× and zinc at 4× to produce an acne scrub. Patients that used the acne scrub noticed an immediate clearing of acne and a definite reduction of redness and a marked improvement in skin appearance.

### Example 5

### Acne Cleanser

A liquid soap formulation was made from the following ingredients: liquid castile soap, imidurea. sodium benzoate, propylene glycol, Vitamin E, glycerine, polysorbate 20, and potassium sorbate. Then, a diluent composition including salicylic acid 5%, trona 2.5% and the following homeopathies Aloe 3×, Silver 4×, Cloves 3×. Lavender 3×. Menthol 3×. Rosemary 3×, and Thyme 4× was incorporated into the liquid soap formulation to obtain a cleanser.

The cleanser left a clean and very soft skin on the patients. The menthol and aloe increased circulation and left the skin cool and refreshed.

### Example 6

### Acne Topical Gel

A blue translucent gel with herbal fragrance was formulated. The gel included' distilled water (deionized water). sodium carboxymethylcellulose, glycine. sodium benzoate, imidurea, potassium sorbate, benzyl alcohol in a base of the following homeopathies: Salicylic Acid 0.2×. Sliver 4×, Zinc 3×, Calendula 3×. Aloe 3×, Aconite 12×. Apis MeI 12×, Cantharis 12×, Acidum Carbolicum 2×, Borax 3×, Natrium Carb (Trona Salt) 2×.

This gel showed astringent and cooling properties. The gel was well absorbed by the skin and did not leave any residue.

### Example 7

### Bar Soap

A vegetable bar soap having a white to off-white color and providing a high full lather that leaves the skin clean and fragrant is prepared. The soap is manufactured by combining 360 grams of coconut oil. 360 grams of olive oil, 600 grams of vegetable shortening. 480 grams water and 200 grams of refined trona salt (sodium carbonate). The oils are heated to 50° C. and set aside. The trona is placed in water and heated to 50° C. and stirred until clear. 15 grams of salicylic acid are added to the heated trona. The heated oils are combined with the trona and salicylic acid mixture and stirred until the soap begins to thicken. 5 grams of Lavender and 5 grams of Lime oil are added and stirred in. The mixture is placed into soap molds and cured for 48 hours.

In some embodiments, the soap can be ground, added to powdered bubble bath and laundry soap mixture to make all natural soaps.

### Example 8

### Bubble Bath

A powdered soap is manufactured and combined with powdered trona or trona salts at 5%, salicylic acid at 1% and the following homeopathies: lavender 3×, orange oil 3×, and chamomile 3×, food colors, sudsing agents sodium laureth sulfate and cocamido-sulfosuccinate and other fragrances. In some embodiments, the powdered soap may be substituted for the sudsing agents listed above to make an all natural bubble bath.

### Example 9

### Laundry Detergent

A powdered soap composed of powdered trona or trona salts at 5%, salicylic acid at 1% and the following homeopathies: lavender 3×, lemon 3×, lime 3×. Food colors, magnesium peroxide, sudsing agents: sodium laureth sulfate and eocamido sulfosuecinate and other fragrances. Powdered soap may be substituted for the sudsing agents listed above to make an all natural laundry detergent.

### Example 10

### Plant Spray

A liquid spray containing trona salts at 1% and salicylic acid at 2% in a base of deionized water, propylene glycol, benzyl alcohol, imidurea, sodium proprionate, and vitamin B1 was formulated. This spray makes flowers and vegetables stay fresh weeks longer.

### Example 11

### Flower Vase Powdered Additive

This example describes a powder that may be added to the water that cut flowers are placed in. The formula may include trona salts at 1%, salicylic acid at 5%, and vitamin B1 at 0.1% with sodium proprionate and sodium benzoate as preservatives. To use, one would add ½ teaspoon per quart of water. This additive makes flowers stay fresh weeks longer.

### Example 12

### Food Freshness Extender

A powder comprising salicylic acid from 1% to 50% salicylic acid and trona at from 1% to 50% that may be placed in the environment of freshly ground or cut meat including beef, pork, poultry and all types of seafood. This powder can be enclosed in breathable packaging such as slotted polyethylene plastic containers or sealed Tyvek film pouches. This powder may be added to water at the ratio of 1% to saturation and poured on absorbent pads and then sealed in polyethylene containers or sealed in Tyvek pouches.

This invention also entails the use of a two component system composed of an aqueous matrix sealed inside a Tyvek pouch that when punctured wets an absorbent pad. This then reacts and releases carbon dioxide and gaseous salicylic into the environment to enhance the freshness of the enclosed foods. Foods may be enclosed in single use packaging or in recirculating refrigeration display cases or even closed walk-in coolers.

### Example 13

### Use of Invention in Maintaining Food Freshness

A retail store in Salt Lake City, Utah allowed the testing of a homeopathic composition having the same component formulation as the spray of Example 1, by placing pouches behind the fresh produce in the display cases and the walk-in coolers. One 70 gram packet was used for every lineal foot in the display cases and placed two packets attached to the discharge vent of each cooler in the walk-in coolers. It was found that the produce losses due to spoilage went from $95 per day to $40 per day over a period of 60 days. Fruit losses went from $34 per day down to $12 per day and other produce went from $57 per day down to $28 per day. On a percentage basis the total losses went from 13% of gross sales down to 3% of gross sales losses during this 60 day period. The packets were changed out every thirty days because gas production has been shown to drop after one month. It was observed that the crates of produce looked just as fresh in the cooler after 5 days as the new produce that was delivered that day.

### Example 14

### Expectorant Cough Syrup

A cherry flavored syrup having a soothing effect on sore throat and containing natural expectorants to break up mucus is prepared. The formula consists of alcohol/sugar syrup with cherry flavor to which the following homeopathies are added: Humulus 4×, Phosphorus 12×. Spongia 5×. Ammonium Bromide 3×. Ephedra 4×, Kali Carb(Trona Salt) 12×, Arsenicum 8×. Anise 4×, Menthol 3×, and Salicylic acid 2×, and Natrium Carb (Trona Salt) 2×.

### Example 15

### Sore Throat Lozenges

A chewable lozenge with an orange flavor is prepared. It consists of the following: fructose, dextrose, sterotex, magnesium stearate, orange flavor, orange food color. citric acid. Vitamin C 100 mg to which the following homeopathics are added: Zinc 5 mg, Silver 4×. Belladonna 12×, Rhus Tox 12×. Sticta 12×. Drosera 6×. Spongia 6×, Cinnaberris 12×. Salicylic Acid 2×, Natrium Carb(Trona Salt) 2×.

### Example 16

### Healing Cream

A healing cream is prepared having the components as listed in the following table:

| Type Of | | | | | | Type of |
|---|---|---|---|---|---|---|
| Ingredient | Ingredient Name | Latin Name | Active Agent | Mg or IU's | | Ingredient |
| | Deionized Water | | | 560 | mg | B |
| | Glycerine DSP | | | 100 | mg | B |
| | Avocado Oil | | | 75 | mg | A |
| Homeopathic | Salicylic Acid | Salicylicum | Same | 50 | mg | B |
| | | Acidum | | | | |
| | Ritachol 2000 | | | 40 | mg | A |
| | Trona Powder | | | 9 | mg | B |
| | Glyceryl Stearate | | | 30 | mg | A |
| Homeopathic | Menthol | Mentholicum | Same | 30 | mg | B |
| | Dimethyl Sulfone | | | 30 | mg | B |
| | Polysorbate 20 | | | 10 | mg | A |
| Homeopathic | Zinc Sulfate | Zincum | Same | 10 | mg | B |
| | | Metalicum | | | | |
| | Capric/Caprylic | | | 25 | mg | A |
| | Triglyceride | | | | | |
| | Cetyl Esters NF | | | 23 | mg | A |
| | Vitamin E Acetate | | | 10 | mg | A |
| | Lecithin | | | 3 | mg | A |
| | Disodium EDTA | | Preservative | 3 | mg | B |
| | Benzyl Alcohol | | Preservative | 3 | mg | A |
| Homeopathic | Silver | Argentum | Silver | 0.1 | mg | B |
| Homeopathic | Arnica Montana 1x | Same | Abrotanin, | 1 | mg | A |
| | | | Absinthol | | | |
| Homeopathic | Aesculus | Same | Aesculin | 1 | mg | A |
| | Hippocastum 1x | | | | | |
| Homeopathic | Berberis Vulgaris 1x | Same | Berberine | 1 | mg | A |
| Homeopathic | Calendula | Same | Calendulin | 1 | mg | A |
| | Officianalis 1x | | | | | |
| Homeopathic | Aloe Vera 1x | Same | Barbaloin | 1 | mg | B |

The cream is prepared by first combining the A ingredients from the table in a jacketed stainless steel kettle and heating to 65° C. The mixture is stirred to prevent burning.

In a similar stainless steel tank the B ingredients from the table are at room temperature. The mixture is stirred with a homogenizing mixer until homogenous.

The A and B ingredients are then combined and stirred until homogenous and a creamy cream is formed. The resulting mixture is cooled overnight and mixed again before filling into dispensing containers.

This product promotes healing and promotes circulation in extremities. Also heals diabetic lesions and bed sores within 4 days. It also restores circulation in feet and restores feeling with 10 to 14 days if applied daily morning and evening to affected area.

### Example 17

### Meat and Vegetable Preservative Powder

The ingredients from the following table are combined and milled to a fine powder. The powder is placed into 70 gram bags of Tyvek and sealed.

| Type of | | | Active | Mg per |
|---|---|---|---|---|
| Ingredient | Ingredient Name | Latin Name | Agent | Gram |
| | Trona Powder | | Bicarbonate | 450 mg |
| Homeopathic | Salicylic Acid | Salicylicum | Same | 450 mg |
| | | Acidum | | |
| Homeopathic | Salvia Officinalis | Same | Camphor | 10 mg |
| Homeopathic | Thyme | Thymus | Thymol | 10 mg |
| | | Serpyllum | | |
| | Chlorella | Same | Chlorophyl | 80 mg |

The bags maybe placed in refrigerator banks at 3 foot intervals or in storage refrigerators at 3 foot intervals. The filled bags will generally last for about 1 month. The composition inbags help vegetables to last 1 to 2 weeks longer, meat and chicken last 1 to 2 weeks longer without spoilage and flowers last 1 week longer.

It should be understood. of course. that the foregoing relates to exemplary embodiments of the invention and that modifications may be made without departing from the spirit and scope of the invention as set forth in the following claims.

What is claimed is:

1. A homeopathic composition for the treatment of skin irritations and other ailments comprising effective amounts of salicylic acid; a member selected from the group consisting of trona and a trona salt; and at least one homeopathic ingredient.

2. A homeopathic composition according to claim 1 wherein the homeopathic composition is contained in a base.

3. The homeopathic composition of claim 2 containing 1% to 10% salicylic acid, 1% to 10% of a member selected from the group consisting of trona and a trona salt, and 1× to 12× of one or more homeopathic ingredients.

4. The homeopathic composition of claim 3 wherein the homeopathic ingredient is a member selected from the group consisting of Aesculus Hippocastum, Berberis Vulgaris, Calendula Officianalis, Aloe Vera, Salvia Officinalis, Thyme, Menthol, Zinc Metallicum and salts thereof, Arnica Montana, Silver and salts, Potassium Chloride, Sodium Chloride, Phosphorus, Sulphur, Manganese Metallicum and salts thereof, Copper Metalicum and salts thereof, Clove, Golden Sea, Witch Hazel, Wintergreen, Frankencense, Myrrh, Eucalptus, White Willow, Rosemary, Lavender, Lemon, Lime, Orange, Arsenicum Album, Arsenicum Trioxide, and Mercury Metalicum and salts thereof.

5. The homeopathic composition of claim 4 wherein the homeopathic ingredient is a member selected from the group consisting of Aesculus Hippocastum, Berberis Vulgaris, Calendula Officianalis, Aloe Vera, Salvia Officinalis, and Thyme.

6. The homeopathic composition of claim 3, wherein the base is selected from the group consisting of hand creams, shaving creams, antibacterial creams, acne cleansers, facial cleaning creams, body creams, toothpastes, acne scrubs, facial cleansers, liquid soaps, liquid detergents, liquid bubble bath soaps, food preservatives, face cleansers, food additives, liquid laundry detergent, food preservation agents, acne topical gels, air refreshers in gel form, food preservation agents, face cleansers, soaps in gel form, bar soaps, capsules, pills, acne lotions, shaving lotions, detergents, plants food additives, and food preservatives.

7. The homeopathic composition of claim 3 wherein the base is a member selected from the group consisting of oral administrating solutions, tablets, and capsules.

8. The homeopathic composition of claim 3 containing trona.

9. The homeopathic composition of claim 3 containing a trona salt.

10. A method for treating a skin irritation or other ailments in a patient, the method comprising the step of topically or orally administering to an affected area a therapeutically effective amount of a homeopathic composition comprising salicylic acid; trona or one of its salts; and at least one homeopathic agent.

11. A method according to claim 10 wherein the homeopathic composition is contained in a base.

12. A method according to claim 11 wherein the composition contains 1% to 10% salicylic acid, 1% to 10% of a member selected from the group consisting of trona and a trona salt, and 1× to 12× of one or more homeopathic ingredients.

13. A method according to claim 12 wherein the homeopathic ingredient administered is a member selected from the group consisting of Aesculus Hippocastum, Berberis Vulgaris, Calendula Officianalis, Aloe Vera, Salvia Officinalis, Thyme, Menthol, Zinc Metallicum and salts thereof, Arnica Montana, Silver and salts, Potassium Chloride, Sodium Chloride, Phosphorus, Sulphur, Manganese Metallicum and salts thereof, Copper Metalicum and salts thereof, Clove, Golden Sea, Witch Hazel, Wintergreen, Frankencense, Myrrh, Eucalptus, White Willow, Rosemary, Lavender, Lemon, Lime, Orange, Arsenicum Album, Arsenicum Trioxide, and Mercury Metalicum and salts thereof.

14. The homeopathic composition of claim 4 wherein the homeopathic ingredient administered is a member selected from the group consisting of Aesculus Hippocastum, Berberis Vulgaris, Calendula Officianalis, Aloe Vera, Salvia Officinalis, and Thyme.

15. The method of claim 12, wherein the patient is a human or an animal.

16. The method of claim 12 wherein the composition administered contains trona.

17. The method of claim 12 wherein the composition administered contains a trona salt.

18. The method of claim 15 wherein the composition is administered topically.

19. The method of claim 15 wherein the composition is administered orally.

20. A method for maintaining the freshness of a product, the method comprising the steps of administering to an environment surrounding the product a homeopathic composition comprising salicylic acid; trona or one of its salts; and at least one homeopathic agent.

21. A method according to claim 20 wherein the product is a member selected from the group consisting of selected from candy, vegetables, flowers, meats and baked goods.

22. The method according to claim 21 wherein the composition administered contains 1% to 10% salicylic acid, 1% to 10% of a member selected from the group consisting of trona and a trona salt, and 1× to 12× of one or more homeopathic ingredients.

23. A method according to claim 22 wherein the homeopathic ingredient administered is a member selected from the group consisting of Aesculus Hippocastum, Berberis Vulgaris, Calendula Officianalis, Aloe Vera, Salvia Officinalis, Thyme, Menthol, Zinc Metallicum and salts thereof, Arnica Montana, Silver and salts, Potassium Chloride, Sodium Chloride, Phosphorus, Sulphur, Manganese Metallicum and salts thereof, Copper Metalicum and salts thereof, Clove, Golden Sea, Witch Hazel, Wintergreen, Frankencense, Myrrh, Eucalyptus, White Willow, Rosemary, Lavender, Lemon, Lime, Orange, Arsenicum Album, Arsenicum Trioxide, and Mercury Metalicum and salts thereof.

24. A method according to claim 23 wherein the homeopathic ingredient administered is a member selected from the group consisting of Aesculus Hippocastum, Berberis Vulgaris, Calendula Officianalis, Aloe Vera, Salvia Officinalis, and Thyme.

25. The method of claim 22 wherein the composition administered contains trona.

26. The method of claim 22 wherein the composition administered contains a trona salt.
